(19) United States
(12) Patent Application Publication
(10) Pub. No.: US 2016/0228344 A1
(43) Pub. Date: Aug. 11, 2016

(54) ANHYDROUS BASE FOR COSMETIC OR DRUG FORMULATIONS

(71) Applicants: ANTHONY ESPOSITO, ROSELLE, NJ (US); MICHAEL A. KNOPF, LONG VALLEY, NJ (US); AMIT PATEL, PINEBROOK, NJ (US); LYUDMILA BEREZNER, BROOKLYN, NY (US); THOMAS SCHAMPER, EAST WINDSOR, NJ (US); WILLIAM C. WOHLAND, SUCCASUNNA, NJ (US)

(72) Inventors: ANTHONY ESPOSITO, ROSELLE, NJ (US); MICHAEL A. KNOPF, LONG VALLEY, NJ (US); AMIT PATEL, PINEBROOK, NJ (US); LYUDMILA BEREZNER, BROOKLYN, NY (US); THOMAS SCHAMPER, EAST WINDSOR, NJ (US); WILLIAM C. WOHLAND, SUCCASUNNA, NJ (US)

(21) Appl. No.: 15/019,308

(22) Filed: Feb. 9, 2016

Related U.S. Application Data
(60) Provisional application No. 62/113,867, filed on Feb. 9, 2015.

(51) Int. Cl. A61K 8/58; A61Q 15/00; A61K 8/26; A61K 8/06; A61K 8/34

(52) U.S. Cl. CPC A61K 8/585; A61K 8/06; A61K 8/345; A61K 8/26; A61Q 15/00; A61K 2800/31

(57) ABSTRACT

Embodiments disclosed herein include a deodorant or antiperspirant base that includes cyclomethicone and propylene glycol dispersed in the cyclomethicone.

### CLAIM OF PRIORITY

This patent application claims the benefit of priority of U.S. Provisional Patent Application Ser. No. 62/113,867, filed Feb. 9, 2015, which is hereby incorporated by reference herein in its entirety.

### FIELD

Inventive embodiments disclosed herein relate to anhydrous base formulations for cosmetic or drug formulations.

### BACKGROUND

Many active cosmetic and drug formulations use large amounts of water to create them. This is due to the wonderful solubility properties of water and the fact that it is an inexpensive ingredient. A major reason for creating an anhydrous carrier system is that some active cosmetic ingredients and drugs are incompatible with water. The ingredients incompatibility can be caused by factors such as insolubility of the ingredients in water and/or instability of the ingredients in water. Another incompatibility could be the instability of the water containing formulas.

Conventional antiperspirant actives, such as ACH or ZAG complexes are soluble in water and are blended with water to make the antiperspirant formulae. Other materials, such as silicone oils or ester oils are added to reduce tackiness. Other conventional formulations include aluminum powders suspended in anhydrous systems.

### SUMMARY

Embodiments disclosed herein include an anhydrous carrier comprising an anhydrous emulsion that includes cyclomethicone and propylene glycol dispersed in the cyclomethicone.

Another embodiment includes a deodorant or antiperspirant base including an emulsion of cyclomethicone and droplets of propylene glycol and a small amount of water less than 1.5%.

Another embodiment is an antiperspirant or deodorant that comprises the anhydrous emulsion of cyclomethicone and propylene glycol dispersed in the cyclomethicone and a moisture absorbing complex.

Embodiments disclosed herein include a deodorant or antiperspirant base comprising an anhydrous emulsion that includes cyclomethicone and propylene glycol where the cyclomethicone is dispersed in the propylene glycol.

Another embodiment includes a deodorant or antiperspirant base including an emulsion of cyclomethicone dispersed in propylene glycol and a small amount of water (less than 1%).

Another embodiment is an antiperspirant or deodorant that comprises the anhydrous emulsion of cyclomethicone dispersed in propylene glycol and a moisture absorbing complex.

### DETAILED DESCRIPTION

Inventive subject matter disclosed herein includes an anhydrous carrier for cosmetic or drug ingredients. This anhydrous carrier contains propylene glycol or higher glycols along with cyclomethicone as the continuous phase/internal phase or vice versa Cosmetic or drug materials can be solubilized or suspended or both soluble and suspended in the carrier phase. Since cyclomethicone and propylene glycol are insoluble together different surfactants and emulsifiers are used to create the needed emulsions. The anhydrous carrier displays improved skin compatibility and efficacy when combined with a moisture absorbing complex, such as is disclosed herein.

Another embodiment includes an anhydrous carrier having an emulsion of cyclomethicone in propylene glycol and a thickening agent dissolved in the propylene glycol. The thickening agent aids in the stability of the anhydrous emulsion.

Another embodiment includes an anhydrous carrier having an emulsion of cyclomethicone in propylene glycol, a sodium hydroxide neutralizing aq. solution. The water has a maximum concentration of 1.0% water by weight and is present due to the sodium hydroxide neutralizing solution and any humidity the base absorbs in processing

One other embodiment includes an anhydrous carrier having an emulsion of cyclomethicone in propylene glycol; thickening agent and a small amount of water.

Another embodiment is an anhydrous carrier used as an antiperspirant or deodorant that includes one of the base emulsion embodiments disclosed herein and a moisture absorbing complex. To make an antiperspirant or deodorant, the moisture control complex is also included in the propylene glycol phase. For some embodiments, the propylene glycol phase includes a small amount of water, due to absorption of humidity. The water is not more than 1% by weight.

A typical formula for this anhydrous carrier is formed by emulsifying propylene glycol into cyclomethicone using a dimethicone copolyol surfactant. One example includes:

| | |
|---|---|
| Cyclomethicone | 5% to 35% |
| Dimethicone Copolyol | 1% to 15% |
| Propylene Glycol | 50% to 94% |

This type of formula usually generates a propylene glycol in cyclomethicone emulsion. The viscosity of this type of emulsion is a function of the volume percent of the internal phase with the external phase of the emulsion. As the internal volume percent increases the viscosity increases.

Optional ingredients can be added to the above formula such as Phenyl Trimethicone (Phenyltris(trimethylsiloxy)silane). Phenyl Trimethicone can help to stabilize the above formula.

| | |
|---|---|
| Cyclomethicone | 5% to 35% |
| Phenyl Trimethicone | 1% to 10% |
| Dimethicone Copolyol | 1% to 15% |
| Propylene Glycol | 40% to 93% |

Through experimentation it was found that the addition of certain cosmetic and or drug ingredients would cause the breakdown of the emulsion immediately or within a few hours after the initial emulsion was formed. To create stable viable emulsions the propylene glycol phase includes:

| | |
|---|---|
| Propylene glycol | 26.78% |
| Triethyl citrate | 0.12% |
| Ethylhexylglycerin | 0.1% |
| Fragrance | 1.0% |
| Moisture Absorption Complex | 4.5% |

A silicone phase includes the following ingredients:

| | |
|---|---|
| Cyclopentasiloxane | 15% |
| Cetyl PEG/PPG 10/1 | 0.5% |

The propylene glycol phase and cyclomethicone phase are combined while stirring. An emulsion formed. Propylene glycol (52%) was added. The system was stable but no increase in viscosity was evident. The Emulsion broke after 18 hours.

To further improve the viability of a robust emulsion thickening agents were added to increase the viscosity of the formula in order to stabilize the emulsion and to give the formula the thickness needed for a Roll-On product. Thickening agents that have been used in this system are fumed silica, Hydroxypropylcellulose, Carbopol, and Ultrathix P-100. The best thickener for these formulas was the Ultrathix P-100.

The base formulations disclosed herein are used, for some embodiments, in conjunction with a moisture absorption complex. In one embodiment, the moisture absorption complex is disclosed in U.S. Pat. No. 8,900,609, which is included herein by reference.

The moisture absorbing complex includes the following: (a) 0.1 to 90% of at least one moisture-absorbing component, (b) 0.1 to 80% of at least one surface-active agent, (c) 0.001 to 20% of at least one electrolyte, and (d) 0 to 50% of at least one solvent and/or at least one vehicle, wherein the moisture-absorbing complex is in the form of a three-dimensional polymer network of said at least one moisture-absorbing component, capable of swelling in contact with water, encapsulated by a coating of said at least one surface-active agent such that the at least one moisture-absorbing component is protected from absorption of water from the base formulation and further wherein the moisture-absorbing complex is emulsified in the base formulation, wherein the moisture-absorbing complex is obtained by premixing the at least one moisture-absorbing component, the at least one surface-active agent, the at least one electrolyte, and, optionally, the at least one solvent and/or at least one vehicle, with stirring, until a substantially uniform or homogeneous mixture is obtained, and further wherein the premixed moisture-absorbing complex is subsequently mixed with the base formulation to produce the moisture-absorbing cosmetic product. The moisture-absorbing components can be added to the formula in situ.

Production of the moisture absorption complex is performed, for one embodiment, as follows:

Example Production of a Moisture-Absorbing Complex

At least one moisture-absorbing component is placed in a clean, dry stainless steel tank equipped with a stirrer. Thereafter, at least one electrolyte is added. With slow stirring, the mixture is heated to a temperature between 50 and 100° C. and kept at this temperature. Subsequently, at least one surface-active agent is slowly added to the tank. While maintaining the batch temperature at 50-100° C., the mixture is stirred continuously for at least another 15 minutes, until a substantially uniform (homogeneous) mixture is obtained which does not include any undissolved raw materials. Depending on the particular ingredients, the complex thus produced has the consistency of a paste, soft solid or hard wax.

The precise quantities of the ingredients depend on the substances being selected.

Typical weight-based ratios are: moisture-absorbing component(s):electrolyte(s):surface-active agent(s) 50:5:45. The weight ratio of moisture-absorbing component(s) to surface-active agent(s) is 1:(0.25-2), preferably 1:(0.5-1.5). The preferred batch temperature for the process depends on the melting points of the ingredients, particularly those of the moisture-absorbing component and surface-active agent, and it varies between 50 and 100° C., particularly between 60 and 90° C. For most ingredients, a batch temperature between 70 and 80° C. is suitable.

When using a solid surface-active agent, a solvent and/or a vehicle is subsequently added to the mixture of moisture-absorbing component, electrolyte and surface-active agent, and stirring is continued until a homogeneous mixture with no undissolved raw material is present.

A defined quantity of each raw material (unprocessed moisture-absorbing component) was placed in a humidity cabinet with a relative humidity of 95-99% and incubated therein for eight weeks. The percent water absorption of each sample was calculated by differential weighing of the sample prior to and after incubation in the humidity cabinet and dividing the weight difference by the sample weight prior to incubation.

Water Absorption Behavior of Various Water Absorbing Components in a Deodorant Base Formulation

The unprocessed moisture-absorbing component (raw material; 5 g each time) was stirred with 95 g of molten deodorant base formulation, 70-80 wt.-% propylene glycol, 20-30 percent by weight cyclomethicone, until to a homogeneous mixture was obtained. The mixture was cooled to room temperature to obtain the shape of a stick.

A defined quantity of each of these mixtures was placed on glass wool in a sealed container including an excess of water and subsequently incubated at 37° C. for 24 hours. A sample of pure deodorant base formulation was incubated in the container in the same way. After careful decanting of the excess water from the container, the percent water absorption was determined by differential weighing of each sample prior to and after water exposure.

The results for a polysaccharide gum from microbial biosynthesis (sclerotium gum from Alban Muller Ind.) and a polysaccharide gum of vegetable origin (blend of xanthan and Cyamopsis tetragonolobus gum (=guar) from TIC Gums, Inc. show that the deodorants containing polysaccharide gums absorb 1.5 times the amount of water compared to a regular deodorant with no polysaccharide additive. It is thus demonstrated that addition of 1 wt.-% of polysaccharide gum provides an additional water storage capacity of 15 to 23% to a regular, hydrous deodorant base formulation.

Water Absorption Behavior of Various Moisture-Absorbing Complexes in a Deodorant Base Formulation

An electrolyte-containing moisture-absorbing complex in accordance with the present invention (complex A) and an electrolyte-free complex (complex B) were produced according to the procedure described above. The compositions of the two complexes are specified in Table 1.

1 g of each complex was stirred with 97.5 g of a molten deodorant base formulation (70-80 wt.-% propylene glycol, 10-15% H₂O, 5-8% cyclomethicone and 1.5 g of an aromatic essence until a homogeneous mixture was obtained. The mixture was cooled to room temperature to obtain the shape of a stick. Consequently, the products obtained included 1 wt.-% of the respective complex and 0.5 wt.-% moisture-absorbing component.

A defined quantity of each of these mixtures was placed on glass wool in a sealed container including an excess of water and subsequently incubated at 37° C. for 24 hours. After careful decanting of the excess water from the container, the percent water absorption was determined by differential weighing of each sample prior to and after water exposure.

The data show that the deodorant with the complexes A and B can absorb more than 0.7 and 0.2 times, respectively, the amount of water compared to a regular deodorant. Moreover, the data demonstrate that the electrolytes have a very favorable influence on the water storage capacity of the absorber material. Ultimately, 1 wt.-% of polysaccharide gum added in the form of the complex according to the invention results in an additional water storage capacity of 360% compared to the regular deodorant base formulation. The example demonstrates that the sweat-absorbing complex of the invention results in a substantially higher water storage capacity compared to the pure moisture-absorbing component in the deodorant.

TABLE 1

| | Complex A | Complex B |
|---|---|---|
| Polysaccharide gum | 50% | 50% |
| (xanthan + Cyamopsis tetragonolobus | | |
| gum) | | |
| Surface-active agent, calculated | 45% | 50% |
| HLB = 7.5 | | |
| (saccharose stearate + sorbitan | | |
| sesquioleate) | | |
| Electrolyte (sodium citrate) | 5% | — |
| Sum | 100% | 100% |

Water Absorption Behavior of Various Sweat-Absorbing Complexes in a Deodorant Base Formulation

Two types of moisture-absorbing complexes (complexes C and D) with the compositions specified in Table 2 (in g or wt.-%) were produced in analogy to Example 5. Essentially, the complexes differ in the levels of electrolyte (sodium citrate). As described above, these complexes were incorporated in a deodorant base formulation to obtain deodorant products having the compositions specified in Table 3 (in g or wt.-%). The ranges specified in Tables 2 and 3 approximately represent the ranges covered in the test series, while the values in parentheses correspond to actual examples. Ultimately, the product containing complex C (Test 76-1) included 0.05 wt.-% electrolyte, and the product containing complex D (Test 76-2) included 0.001 wt.-% electrolyte. Similarly, a comparative example with no complex and thus no electrolyte was produced (Test 76-3).

The samples were weighed on glass wool in a sealed container. Following addition of water to the container, the samples were incubated at 37° C. for 24 hours. After careful decanting of the water, the samples were re-weighed, and the water absorption was determined by differential weighing prior to and after water exposure.

Although the result shows a somewhat higher water absorption of the high-electrolyte sample, Test 76-1 (complex C), than the Test 76-2 (complex D) sample containing less electrolyte, this difference is less significant (according to the “Student T test” p=0.28 and n=5). On the other hand, the water absorption of the complex-containing samples Test 76-1 and 76-2 is significantly higher (p<0.01, n=5) than that of the complex-free sample Test 76-3 (pure deo base).

TABLE 2

| | |
|---|---|
| Complex C | Complex D |

| | | | | |
|---|---|---|---|---|
| Propylene glycol | 30-35 | (33.4) | 33-40 | (36.6) |
| Sodium citrate | 3-5 | (3.3) | 0.05-0.1 | (0.07) |
| Natural cotton | 0.1-0.5 | (0.2) | 0.1-0.5 | (0.2) |
| Xanthan + guar gum | 30-35 | (33.1) | 30-35 | (33.1) |
| Saccharose stearate | 8-15 | (10) | 8-15 | (10) |
| Sorbitan sesquioleate | 18-23 | (20) | 18-23 | (20) |

| | | |
|---|---|---|
| Sum | 100 | 100 |

TABLE 3

| | Test 76-1 | | Test 76-2 | | | |
|---|---|---|---|---|---|---|
| | complex C | | complex D | | Test 76-3 | |
| | in deo base | | in deo base | | deo base | |
| Propylene | 65-70 | (67.8) | 65-70 | (67.8) | 67-72 | (69.3) |
| glycol | | | | | | |
| Water. Deion- | 17-23 | (19) | 17-23 | (19) | 17-23 | (19) |
| ized | | | | | | |
| Triclosan | 0.1-0.5 | (0.3) | 0.1-05 | (0.3) | 0.1-0.5 | (0.3) |
| Sodium | 7-12 | (9) | 7-12 | (9) | 7-12 | (9) |
| stearate | | | | | | |
| Stearic acid | 0.5-1 | (0.75) | 0.5-1 | (0.75) | 0.5-1 | (0.75) |
| Water abs. | 1-2 | (1.5) | — | | — | |
| complex C | | | | | | |
| (Tab. 2) | | | | | | |
| Water abs. | — | | 1-2 | (1.5) | — | |
| complex D | | | | | | |
| (Tab. 2) | | | | | | |
| Aromatic | a.r. | | a.r. | | a.r. | |
| essence | | | | | | |
| Allantoin | 0.1-1 | (0.1) | 0.1-1 | (0.1) | 0.1-1 | (0.1) |

| | | | |
|---|---|---|---|
| Sum | 100 | 100 | 100 |

In various embodiments, the inventive composition or method can be any one of any of the combinations and/or sub-combinations of the above-listed embodiments.

### Exemplary Roll-On Antiperspirant Formula-1:

| Ingredient | w/w % Concentration |
|---|---|
| Phase 1 | |
| Propylene Glycol | 26.78 |
| Triethyl Citrate | 0.120 |
| Tocopheral | 0.100 |
| Ethylhexylglycerin | |
| Water | |
| Fragrance | 1.00 |
| Moisture Absorption Complex | 4.500 |
| Phase 2 | |
| Dow Corning 245 Fluid | 15.00 |
| Abil EM-90 | 0.500 |
| Phase 3 | |
| Propylene Glycol | 52.00 |

The Moisture Absorption Complex includes the following:

| Ingredient | w/w % Concentration |
|---|---|
| Sodium citrate | 0.0700 |
| Palmitic Acid | 0.0900 |
| Gossypium Herbaceum (Cotton) Powder | 0.2000 |
| Sucrose | 0.2000 |
| Stearic Acid | 0.2100 |
| Water | 0.5000 |
| Xanthan gum | 8.2830 |
| Sucrose Stearate | 9.0000 |
| Sorbitan Sesquioleate | 20.0000 |
| Cyamopsis Tetragonoloba (Guar) Gum | 24.8480 |
| Propylene Glycol | 36.5990 |

The Dow Corning 245 Fluid includes Cyclotetrasiloxane in a concentration of 0.7500 w/w % of the Dow Corning 245 fluid; Cyclohexasiloxane in a concentration of 1.2500 w/w %; and 98.000 w/w % of Cyclopentasiloxane.

The Abil EM-90 includes Cetyl PEG/PPG-10/1 Dimethicone in a concentration of 96.8943° % by weight of the Abil EM-90; Pentaeiythrityl Tetra-DI-T-Butyl Hydroxy-Hydrocinnamate in a concentration of 0.0050 w/w %; 1-Hexadecene in a concentration of 3.0000 w/w %; Propylene Oxide in a concentration of 0.0001 w/w %; Ethylene Oxide in a concentration of 0.0001 w/w %, 1,4-Dioxane in a concentration of 0.0005 w/w % and 0.1000 in a concentration of 0.1000 w/w %.

The reference to Tocopheral, Ethylhexylglycerin, and Water in a combined concentration of 0.100 w/w % of the antiperspirant formulation refers to a formulation having a product name of Sensiva SC 50, made by Schulke.

### Exemplary Roll-On Antiperspirant Formula-2

| Ingredient | w/w % Concentration |
|---|---|
| Phase 1 | |
| Propylene Glycol | 60.827 |
| Triethyl Citrate | 0.120 |
| Tocopheral | 0.100 |
| Ethylhexylglycerin | |
| Water | |
| Fragrance | 1.00 |
| Phase 2 | |
| Propylene glycol | 20.000 |
| Xanthan Gum + Guar Gum | 1.500 |
| Sisterna Sucrose Ester | 0.450 |
| Phase 3 | |
| Sodium citrate | 0.003 |
| Water | 0.500 |
| Phase 4 | |
| Dow Corning 245 Fluid | 15.000 |
| Abil EM-90 | 0.500 |

The Sisterna Sucrose Ester includes 0.9000 Palmitic Acid w/w % of the Sisterna Sucrose Ester; 2.000 w/w % of Sucrose; 2.100 w/w % of Stearic Acid; 5.000 w/w % of water and 90.000 w/w % of Sucrose Stearate.

### Exemplary Roll-On Antiperspirant Formula-3

| Ingredient | w/w % |
|---|---|
| Phase 1 | |
| Propylene Glycol | 62.280 |
| Gel Base of Propylene Glycol (97.5%) and Carbomer (2.50%) | 16.000 |
| Triethyl Citrate | 0.120 |
| Tocopheral | 0.100 |
| Ethylhexylglycerin | |
| Water | |
| Fragrance | 1.000 |
| Phase 2 | |
| Dow Corning 245 Fluid | 15.000 |
| Moisturre Absorption complex | 4.5000 |
| Abil EM-90 | 1.000 |

### Exemplary Roll-On Antiperspirant Formula-4

| Phase 1 | |
|---|---|
| Propylene Glycol | 66.280 |
| Gel Base of Propylene Glycol (97.5%) and Carbomer (2.50%) | 12.000 |
| Triethyl Citrate | 0.120 |
| Tocopheral | 0.100 |
| Ethylhexylglycerin | |
| Water | |
| Fragrance | 1.000 |
| Phase 2 | |
| Dow Corning 245 Fluid | 15.000 |
| Moisturre Absorption complex | 4.5000 |
| Abil EM-90 | 1.000 |

### Exemplary Roll-On Antiperspirant Formula-5

| Ingredient | w/w % Concentration |
|---|---|
| Phase 1 | |
| Propylene Glycol | 78.480 |
| Triethyl Citrate | 0.120 |
| Tocopheral | 0.100 |
| Ethylhexylglycerin | |
| Water | |
| Fragrance | 1.00 |
| Carbomer | 0.300 |
| Phase 2 | |
| Moisture Absorption Complex | 4.500 |
| Dow Corning 245 Fluid | 15.00 |
| Abil EM-90 | 0.500 |

### Exemplary Roll-On Antiperspirant Formula-6

| Ingredient | w/w % Concentration |
|---|---|
| Phase 1 | |
| Propylene Glycol | 76.680 |
| Triethyl Citrate | 0.120 |
| Tocopheral | 0.100 |
| Ethylhexylglycerin | |
| Water | |
| Fragrance | 1.00 |
| Silica | 1.600 |
| Phase 2 | |
| Moisture Absorption Complex | 4.500 |
| Dow Corning 245 Fluid | 15.00 |
| Abil EM-90 | 1.00 |

### Exemplary Roll-On Antiperspirant Formula-7

| Ingredient | w/w % Concentration |
|---|---|
| Phase 1 | |
| Propylene Glycol | 75.780 |
| Triethyl Citrate | 0.120 |
| Tocopheral | 0.100 |
| Ethylhexylglycerin | |
| Water | |
| Fragrance | 1.00 |
| Silica | 2.500 |
| Phase 2 | |
| Moisture Absorption Complex | 4.500 |
| Dow Corning 245 Fluid | 15.00 |
| Abil EM-90 | 1.00 |

### Exemplary Roll-On Antiperspirant Formula-8

| Ingredient | w/w % Concentration |
|---|---|
| Phase 1 | |
| Propylene Glycol | 75.280 |
| Triethyl Citrate | 0.120 |
| Tocopheral | 0.100 |
| Ethylhexylglycerin | |
| Water | |
| Fragrance | 1.00 |
| Moisture Absorption Complex | 4.500 |
| Phase 2 | |
| Dow Corning 245 Fluid | 15.00 |
| Abil EM-90 | 4.00 |

### Exemplary Roll-On Antiperspirant Formula-9

| Ingredient | w/w % Concentration |
|---|---|
| Phase 1 | |
| Propylene Glycol | 76.780 |
| Triethyl Citrate | 0.120 |
| Tocopheral | 0.100 |
| Ethylhexylglycerin | |
| Water | |
| Fragrance | 1.00 |
| Moisture Absorption Complex | 4.500 |
| Phase 2 | |
| Dow Corning 245 Fluid | 10.00 |
| Formulation Aid | 7.500 |

The formulation aid includes Cyclotetrasiloxane in a concentration of 0.900% w/w of the formulation aid; water in a concentration of 1.000% w/w; 10.500 w/w of PEG/PPG-18/18 Dimethicone and 87.600% w/w of cyclopentasiloxane.

### Exemplary Roll-On Antiperspirant Formula-10

| Ingredient | w/w % Concentration |
|---|---|
| Phase 1 | |
| Propylene Glycol | 74.880 |
| Triethyl Citrate | 0.120 |
| Tocopheral | 0.100 |
| Ethylhexylglycerin | |
| Water | |
| Fragrance | 1.00 |
| Moisture Absorption Complex | 4.500 |
| Ultrathix P-100 | 0.400 |
| 20% Sodium Hydroxide | 0.100 |
| Phase 2 | |
| Dow Corning 245 Fluid | 15.00 |
| Abil EM-90 | 3.000 |
| Silica | 0.900 |

The Ultrathix P-100 includes acrylic acid/VP crosspolymer in a concentration of 98.9500% w/w of the Ultrathix P-100; heptane in a concentration of 0.9900% w/w; acrylic acid in a concentration of 0.0500% w/w; and N-vinyl pyrollidone in a concentration of 0.0100% w/w.

### Exemplary Roll-On Antiperspirant Formula-11

| Ingredient | w/w % Concentration |
|---|---|
| Phase 1 | |
| Propylene Glycol | 79.78 |
| Triethyl Citrate | 0.120 |
| Tocopheral | 0.100 |
| Ethylhexylglycerin | |
| Water | |
| Fragrance | 1.00 |
| Phase 2 | |
| Dow Corning 245 Fluid | 15.00 |
| Abil EM-90 | 4.000 |

### Exemplary Roll-On Antiperspirant Formula-12

| Ingredient | w/w % Concentration |
|---|---|
| Phase 1 | |
| Propylene Glycol | 81.280 |
| Triethyl Citrate | 0.120 |
| Tocopheral | 0.100 |
| Ethylhexylglycerin | |
| Water | |
| Fragrance | 1.00 |
| Phase 2 | |
| Dow Corning 245 Fluid | 10.00 |
| Formulation Aid | 7.500 |

### Exemplary Roll-On Antiperspirant Formula-13

| Ingredient | w/w % Concentration |
|---|---|
| Phase 1 | |
| Propylene Glycol | 81.780 |
| Triethyl Citrate | 0.120 |
| Tocopheral | 0.100 |
| Ethylhexylglycerin | |
| Water | |
| Fragrance | 1.00 |
| Phase 2 | |
| Dow Corning 245 Fluid | 10.00 |
| Phenyl Trimethicone | 1.00 |
| Abil EM-90 | 1.00 |
| Moisture Absorption Complex | 4.500 |
| Dow Corning Formulation Aid | 0.500 |

The Dow Corning Formulation Aid includes PEG-10 dimethicone in a concentration of 99.90% of the Formulation Aid concentration; and tocopheral in a concentration of 0.100% w/w.

### Exemplary Roll-On Antiperspirant Formula-14

| Ingredient | w/w % Concentration |
|---|---|
| Phase 1 | |
| Propylene Glycol | 80.880 |
| 20% Sodium Hydroxide Solution | 0.300 |
| ULTRATHIX P-100 | 0.700 |
| Triethyl Citrate | 0.120 |
| Tocopheral | 0.100 |
| Ethylhexylglycerin | |
| Water | |
| Fragrance | 1.00 |
| Moisture Absorption Complex | 4.500 |
| Silicone Phase | |
| Dow Corning 245 Fluid | 10.00 |
| Phenyl Trimethicone | 1.000 |
| Abil EM-90 | 1.00 |
| Neutralization Phase | |
| 20% Sodium Hydroxide Solution | 0.400 |

### Exemplary Roll-On Antiperspirant Formula-15

| Ingredient | w/w % Concentration |
|---|---|
| Phase 1 | |
| Propylene Glycol | 77.517 |
| 20% Sodium Hydroxide Solution | 0.100 |
| ULTRATHIX P-100 | 0.400 |
| Sodium Citrate | 0.003 |
| Sisterna Sucrose Ester | 0.450 |
| Triethyl Citrate | 0.120 |
| Tocopheral | 0.100 |
| Ethylhexylglycerin | |
| Water | |
| Fragrance | 1.00 |
| Sorbitan Sesquioleate | 0.900 |
| Guar Gum (75%)/XanthanGum (25%) | 1.500 |
| Silica | 1.600 |
| Gossypium Herbaceum (Cotton) Powder | 0.0100 |
| Silicone Phase | |
| Dow Corning 245 Fluid | 15.00 |
| Abil EM-90 | 1.00 |
| Neutralization Phase | |
| 20% Sodium Hydroxide Solution | 0.300 |

### Exemplary Roll-On Antiperspirant Formula-16

| Ingredient | w/w % Concentration |
|---|---|
| Propylene Glycol | 77.527 |
| 20% Sodium Hydroxide Solution | 0.100 |
| ULTRATHIX P-100 | 0.400 |
| Sodium Citrate | 0.003 |
| Sisterna Sucrose Ester | 0.450 |
| Triethyl Citrate | 0.120 |
| Tocopheral | 0.100 |
| Ethylhexylglycerin | |
| Water | |
| Fragrance | 1.00 |
| Sorbitan Sesquioleate | 0.900 |
| Guar Gum (75%)/XanthanGum (25%) | 1.500 |
| Hydrated Silica | 1.600 |
| Silicone Phase | |
| Dow Corning 245 Fluid | 15.00 |
| Abil EM-90 | 1.00 |
| Neutralization Phase | |
| 20% Sodium Hydroxide Solution | 0.300 |

While the invention has been described and exemplified in sufficient detail for those skilled in this art to make and use it, various alternatives, modifications, and improvements will be apparent to those skilled in the art without departing from the spirit and scope of the claims.

All patents and publications referred to herein are incorporated by reference to the same extent as if each individual publication was specifically and individually indicated to be incorporated by reference.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cosmetic or drug formulations base comprising an anhydrous emulsion of cyclomethicone in propylene glycol.

2. A cosmetic or drug formulations base, comprising an anhydrous emulsion of propylene glycol in cyclomethicone.

3. A cosmetic or drug formulations base comprising an anhydrous emulsion of propylene glycol and cyclomethicone where the emulsifying agent is a dimethicone copolyol.

4. A cosmetic or drug formulations base comprising an anhydrous emulsion of propylene glycol and cyclomethicone where the emulsifying agent is a dimethicone copolyol where the emulsion is stabilized and the viscosity controlled by using thickening agents.

5. A cosmetic or drug formulations base comprising an anhydrous emulsion of cyclomethicone and propylene glycol dispersed in the cyclomethicone and a moisture absorbing complex.

6. The cosmetic or drug formulations base of claim 1, wherein the base is a roll on.

7. The cosmetic or drug formulations base of claim 2, wherein the base is a roll on.

8. The cosmetic or drug formulations base of claim 3, wherein the base is a roll on.

9. The cosmetic or drug formulations base of claim 4, wherein the base is a roll on.

10. The cosmetic or drug formulations base of claim 1, wherein the base is a spray.

11. The cosmetic or drug formulations base of claim 2, wherein the base is a spray.

12. The cosmetic or drug formulations base of claim 3, wherein the base is a spray.

13. The cosmetic or drug formulations base of claim 4, wherein the base is a spray.

14. The cosmetic or drug formulations base of claim 1, wherein the moisture absorbing complex has a concentration of 4.5% by weight of a deodorant or antiperspirant.

15. A deodorant or antiperspirant comprising the cosmetic or drug formulations base of claim 1, free of aluminum salts.

16. A deodorant or antiperspirant comprising the cosmetic or drug formulations base of claim 2, free of aluminum salts.
